SEQUENCE LISTING (4 sequences)

SEQ ID NO: 1 - protein, 1273 aa, synthetic construct
  REGION 1..1273: note = Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1242 aa, synthetic construct
  REGION 1..1242: note = Synthetic
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 3 - protein, 676 aa, synthetic construct
  REGION 1..676: note = Synthetic
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYASGKRSNTTGKLIWKVNPEIDTTIGEWAFWETKKNLTRKIRSEELSFTAVSNGPKNISGQSPARTSSDPETNTTNEDHKIMASENSSAMVQVHSQGRKAAVSHLTTLATISTSPQPPTTKTGPDNSTHNTPVYKLDISEATQVGQHHRRADNDSTASDTPPATTAAGPLKAENTNTSKSADSLDLATTTSPQNYSETAGNNNTHHQDTGEESASSGKLGLITNTIAGVAGLITGGRRTRREVIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 4 - protein, 676 aa, synthetic construct; identical to SEQ ID NO: 3
  REGION 1..676: note = Synthetic

(19) United States
(12) Patent Application Publication
(10) Pub. No.: US 2023/0257719 A1
(43) Pub. Date: Aug. 17, 2023

(54) Production of High Titer Recombinant Vesicular Stomatitis Virus in Suspension Cell Culture

(71) Applicant: Regeneron Pharmaceuticals, Inc., Tarrytown, NY (US)

(72) Inventors: Andrew Tustian, Millwood, NY (US); Zhe Zhang, Scarsdale, NY (US)

(21) Appl. No.: 18/108,417

(22) Filed: Feb. 10, 2023

Related U.S. Application Data
(60) Provisional application No. 63/309,109, filed on Feb. 11, 2022.

(51) Int. Cl. C12N 7/04; C12N 15/85; C07K 14/005

(52) U.S. Cl. CPC C12N 7/04; C12N 15/85; C07K 14/005; C12N 2760/20222; C12N 2500/95; C12N 2760/14122; C12N 2523/00

(57) ABSTRACT

Methods for the production of replication-incompetent recombinant vesicular stomatitis virus (rVSV) in suspension cell culture are disclosed. In some embodiments, the methods include inoculating a suspension cell culture medium with packaging cells, transfecting the packaging cells with a plasmid comprising a nucleic acid molecule encoding a viral envelope glycoprotein, introducing rVSV, devoid of a gene encoding a functional envelope glycoprotein, into the suspension cell culture medium, and isolating rVSV produced from the packaging cells with the viral envelope glycoprotein incorporated into its viral envelope.

### CROSS-REFERENCE TO RELATED APPLICATION

This application claims the benefit under 35 USC § 119(e) of U.S. Provisional Application No. 63/309,109, filed Feb. 11, 2022, which is incorporated herein by reference in its entirety for all purposes.

### REFERENCE TO A SEQUENCE LISTING

This application incorporates by reference a computer readable Sequence Listing in ST.26 XML format, titled 10976US01_Sequence, created on Feb. 10, 2023 and containing 9,944 bytes.

### FIELD OF THE INVENTION

The present invention relates to the production of recombinant viral particles, and in particular to methods for producing replication-incompetent recombinant vesicular stomatitis virus (rVSV) comprising a viral envelope glycoprotein produced in a transfected packaging cell.

### BACKGROUND

The entry of all enveloped viruses requires a membrane fusion event that is mediated by one or more viral glycoproteins found on the surface of the lipid envelope of the virus. These envelope glycoproteins are responsible for binding virus to the cell surface and for inducing fusion of the viral envelope with either the plasma membrane of the host cell, or with an internal membrane following endocytosis of the virion.

Vesicular stomatitis virus (VSV) is a prototypic nonsegmented, negative-stranded RNA virus that belongs to the family Rhabdoviridae. VSV has been used extensively to study virus entry, replication and assembly due to its broad host range and robust replication properties in a wide variety of mammalian and insect cells. One of the remarkable properties of VSV is that VSV virions are not particularly selective regarding the type of membrane protein that can be incorporated into the viral envelope. Early studies in which cells were coinfected with VSV and other enveloped viruses demonstrated that VSV forms pseudotypes readily. A pseudotype has the envelope protein of the heterologous virus assembled into the VSV membrane. The ability to form pseudotypes is likely due to the mechanism of VSV budding, which has been shown not to require VSV G protein. Studies on VSV assembly led to the creation of a recombinant VSV in which the glycoprotein (G) gene was deleted. This recombinant (rVSV-ΔG) has been used to produce VSV pseudotypes containing the envelope glycoproteins of heterologous viruses. (Whitt, J Virol Methods, 169(2):365-374, 2010).

Production of rVSV has relied primarily on anchorage-dependent cell cultivation techniques. However, scale-up of cell production using adherent cell-based manufacturing presents a number of challenges as attachment-dependent culture processing generally requires more steps and time than suspension culture, resulting in more labor intensive and costly production. Thus, there remains a need for the development of scalable propagation methods where yields of recombinant VSV particles are sufficient to be of use in large-scale manufacture.

### BRIEF SUMMARY OF THE INVENTION

The present disclosure is directed to methods for the production of recombinant vesicular stomatitis virus (rVSV) in suspension cell culture that yield viral titers comparable to adherent cell culture processes. The methods can be used, for example, to produce replication incompetent rVSV with envelope glycoproteins of any one of a variety of enveloped viruses (e.g., SARS-CoV-2 or Ebola) for use as a pseudovirus in, e.g., viral antibody neutralization assays, or for use in vector-based vaccines.

In one aspect, the present disclosure provides a method of producing replication-incompetent recombinant vesicular stomatitis virus (rVSV), comprising: (a) inoculating a suspension cell culture medium with a plurality of packaging cells; (b) transfecting the packaging cells in the suspension cell culture medium with a plasmid comprising a nucleic acid molecule encoding a viral envelope glycoprotein to produce a population of packaging cells expressing the viral envelope glycoprotein; (c) introducing rVSV into the suspension cell culture medium at a multiplicity of infection (MOI) rate of from 0.001 to 3 to infect the population of packaging cells expressing the viral envelope glycoprotein, wherein the rVSV has been engineered such that the rVSV does not express a functional envelope glycoprotein; and (d) isolating rVSV produced from the population of packaging cells from 15 to 65 hours post infection, wherein the viral envelope glycoprotein in incorporated into the viral envelope of the isolated rVSV.

In some embodiments, the MOI is from 0.001 to 1.0. In some embodiments, the MOI is from 0.005 to 0.05. In some cases, the MOI is from 0.008 to 0.012. In some cases, the MOI is about 0.01. In some cases, the MOI is from 0.01 to 2. In some cases, the MOI is from 0.01 to 1. In some cases, the MOI is from 0.8 to 1.2.

In some embodiments, the rVSV produced from the packaging cells is isolated from 40 to 50 hours post infection. In some cases, the rVSV produced from the packaging cells is isolated from 43 to 47 hours post infection. In some cases, the rVSV produced from the packaging cells is isolated from 20 to 30 hours post infection. In some cases, the rVSV produced from the packaging cells is isolated from 22 to 26 hours post infection.

In some embodiments, the packaging cells are mammalian cells. In some cases, the packaging cells are primate cells. In some cases, the packaging cells are human cells. In some embodiments, the packaging cells are human embryonic kidney (HEK) cells. In some cases, the HEK cells are HEK293 cells. In some embodiments, the HEK293 cells are HEK293F cells, HEK293T cells, HEK293SF cells, or HEK293S cells. In some cases, the HEK293 cells are HEK293F cells.

In some embodiments, the suspension cell culture medium is a serum-free and protein-free medium.

In some embodiments, the isolated rVSV is produced at an infectious titer of at least 1×10⁶ plaque forming units (pfu)/mL. In some cases, the isolated rVSV is produced at an infectious titer of at least 1×10⁷ pfu/mL. In some cases, the isolated rVSV is produced at an infectious titer of at least 1×10⁸ pfu/mL. In some cases, the isolated rVSV is produced at an infectious titer of at least 5×10⁸ pfu/mL. In some cases, the isolated rVSV is produced at an infectious titer of at least 1×10⁹ pfu/mL. In some cases, the isolated rVSV is produced at a titer equivalent to the titer produced in an adherent cell-based control. In some cases, the isolated rVSV is produced at a titer at least 2 fold greater than the titer produced in an adherent cell-based control. In some cases, the isolated rVSV is produced at a titer at least 3 fold greater than the titer produced in an adherent cell-based control.

In some embodiments, the viral envelope glycoprotein is a vesicular stomatitis virus G protein. In some embodiments, the viral envelope glycoprotein is a class I fusion protein. In some embodiments, the viral envelope glycoprotein is a class II fusion protein. In some embodiments, the viral envelope glycoprotein is a class III fusion protein. In some embodiments, the viral envelope glycoprotein is a coronavirus spike protein. In some cases, the coronavirus spike protein is a spike protein of SARS-CoV-2. In some cases, the coronavirus spike protein comprises the amino acid sequence of SEQ ID NO: 1. In some cases, the coronavirus spike protein comprises the amino acid sequence of SEQ ID NO: 2. In some embodiments, the viral envelope glycoprotein is an ebola virus glycoprotein. In some cases, the ebola virus glycoprotein comprises the amino acid sequence of SEQ ID NO: 3 or SEQ ID NO: 4.

In some embodiments, the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses.

In some embodiments of the method, transfecting the packaging cells comprises use of a polyethylenimine transfection reagent. In some embodiments of the method, transfecting the packaging cells comprises uses of a lipofectamine transfection reagent or the proprietary transfection reagent FectoVIR®-AAV (Polyplus, France) transfection reagent.

In some embodiments, the cell culture medium comprises a cell density of at least 3×10⁶ packaging cells/mL when transfecting the packaging cells.

In some embodiments of the method, transfecting the packaging cells comprises use of a DNA concentration of at least 1.5 μg/mL. In some embodiments of the method, transfecting the packaging cells comprises use of a transfection reagent:DNA ratio of from about 2 to about 3. In some embodiments of the method, transfecting the packaging cells comprises use of a transfection reagent:DNA ratio of about 2.5.

In some embodiments, the suspension cell culture medium is maintained at a temperature during inoculation and transfection, and the temperature of the suspension cell culture medium is reduced to a lower temperature when introducing rVSV into the suspension cell culture medium. In some cases, the temperature is about 37° C. In some cases, the reduced temperature is from about 32° C. to about 36.5° C. In some cases, the reduced temperature is about 35.5° C. In some cases, the reduced temperature is about 34° C.

In some embodiments of the methods discussed herein, the suspension cell culture medium is not exchanged prior to introducing rVSV into the suspension cell culture medium.

In one aspect, the present disclosure provides a method of producing replication-incompetent recombinant vesicular stomatitis virus (rVSV), comprising: (a) inoculating a suspension cell culture medium with a plurality of HEK293 cells, wherein the suspension cell culture medium is at a temperature of about 37° C.; (b) transfecting the HEK293 cells in the suspension cell culture medium with a polyethylenimine transfection reagent and a plasmid comprising a nucleic acid molecule encoding a viral envelope glycoprotein to produce a population of packaging cells expressing the viral envelope glycoprotein; (c) reducing the temperature of the suspension cell culture medium to about 34° C. and introducing rVSV into the suspension cell culture medium at a multiplicity of infection (MOI) rate of about 0.01 to infect the population of HEK293 cells expressing the viral envelope glycoprotein, wherein the rVSV has been engineered such that the rVSV does not express a functional envelope glycoprotein; and (d) isolating rVSV produced from the population of packaging cells from 40 to 48 hours post infection, wherein the viral envelope glycoprotein in incorporated into the viral envelope of the isolated rVSV.

In one aspect, the present disclosure provides a method of producing replication-incompetent recombinant vesicular stomatitis virus (rVSV), comprising: (a) inoculating a suspension cell culture medium with a plurality of HEK293 cells, wherein the suspension cell culture medium is at a temperature of about 37° C.; (b) transfecting the HEK293 cells in the suspension cell culture medium with a polyethylenimine transfection reagent and a plasmid comprising a nucleic acid molecule encoding a viral envelope glycoprotein to produce a population of packaging cells expressing the viral envelope glycoprotein; (c) reducing the temperature of the suspension cell culture medium to about 34° C. and introducing rVSV into the suspension cell culture medium at a multiplicity of infection (MOI) rate of about 1 to infect the population of HEK293 cells expressing the viral envelope glycoprotein, wherein the rVSV has been engineered such that the rVSV does not express a functional envelope glycoprotein; and (d) isolating rVSV produced from the population of packaging cells from 20 to 28 hours post infection, wherein the viral envelope glycoprotein in incorporated into the viral envelope of the isolated rVSV.

In some embodiments, the viral envelope glycoprotein is a class I fusion protein, a class II fusion protein, or a class III fusion protein. In some embodiments, the viral envelope glycoprotein is a coronavirus spike protein. In some cases, the coronavirus is SARS-CoV-2. In some embodiments, the viral envelope glycoprotein is an ebola virus glycoprotein. In some embodiments, the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses.

In some embodiments, the cell culture medium comprises a cell density of at least 3×10⁶ HEK293 cells/mL when transfecting the HEK293 cells.

In some embodiments of the method, transfecting the HEK293 cells comprises use of a DNA concentration of at least 1.5 μg/mL. In some embodiments of the method, transfecting the HEK293 cells comprises use of a transfection reagent:DNA ratio of about 2.5.

In some embodiments, the HEK293 cells are HEK293F cells, HEK293T cells, HEK293SF cells, or HEK293S cells. In some cases, the HEK293 cells are HEK293F cells.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

Other embodiments will become apparent from a review of the ensuing detailed description.

### BRIEF DESCRIPTION OF THE DRAWINGS

FIG. 1 illustrates an exemplary suspension cell culture production schematic. In the illustrated example, recombinant VSV in which the glycoprotein (G) gene was deleted (rVSV-ΔG) is introduced into a suspension cell culture of packaging cells transfected with a plasmid comprising a nucleic acid molecule encoding the VSV-G glycoprotein such that upon virus replication and budding, the rVSV-ΔG incorporates the VSV-G glycoprotein into the viral envelope.

FIG. 2 illustrates additional parameters (from top to bottom at Day 2: temperature, stir speed, pH, and dissolved oxygen) of an exemplary production process in a small-scale bioreactor with the parameters set forth in Table 1 (Example 1).

FIG. 3 illustrates varying parameters and the resulting viral titers at 24, 33 and 48 hours post infection (hpi) in a control sample and seven samples in which a single parameter was varied. The highlighted cells shown the parameter changed for each respective sample. Recombinant VSV-ΔG-Fluc/GFP stock virus (titer=1.59E9) was used for infections. A comparator adherent cell system yielded a viral titer of 9.3E8 pfu/mL.

FIG. 4 illustrates time course titer data (plaque-forming units/mL as a function of time (hours post infection)) corresponding to varying production parameters (e.g., transfection, infection, culture, and harvest conditions) in the fifteen different runs shown in Table 2 (Example 2).

FIGS. 5A and 5B illustrate the effect on viral titer (pfu/mL) of varying parameters of a suspension cell culture production process, including: (i) whether to not to perform a media exchange prior to infection; (ii) multiplicity of infection (MOI) rate; (iii) type of transfection reagent; (iv) temperature shift at time of infection; and (v) harvest time at hours post infection (hpi).

FIG. 6 illustrates an exemplary suspension cell culture production schematic for producing a pseudotyped recombinant VSV (e.g., with a coronavirus spike glycoprotein in place of the VSV-G glycoprotein. In the illustrated example, recombinant VSV in which the glycoprotein (G) gene was deleted (rVSV-ΔG) is introduced into a suspension cell culture of packaging cells transfected with a plasmid comprising a nucleic acid molecule encoding a coronavirus spike glycoprotein such that upon virus replication and budding, the rVSV-ΔG incorporates the coronavirus spike glycoprotein into the viral envelope.

FIG. 7 illustrates the results of a luciferase assay showing the production of spike glycoprotein pseudotyped rVSV (VSV-Spike) at titers more than 3 fold greater than the titers produced by an adherent cell culture control (adherent control D614G) and a mock transfection (background). As shown, at 24 hours post infection, the adherent control showed a titer of 1430 RLUs, while the VSV-Spike showed a titer of about 5300 RLUs.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “include,” “includes,” and “including,” are meant to be non-limiting and are understood to mean “comprise,” “comprises,” and “comprising,” respectively.

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Selected Abbreviations

VSV—vesicular stomatitis virus

rVSV—recombinant vesicular stomatitis virus

rVSV-ΔG—recombinant vesicular stomatitis virus with a deleted glycoprotein (G) gene

VSV-G—envelope glycoprotein (G) of vesicular stomatitis virus

HEK cell—human embryonic kidney cell

PFU—plaque forming units

MOI—multiplicity of infection

VLP—virus like particle

HPI—hours post infection

SARS-CoV-2—serious acute respiratory syndrome coronavirus 2

### Definitions

“VSV” refers to any strain of VSV or mutant forms of VSV, including rVSV. VSV encompasses replication-incompetent VSV, such as, VSV lacking G glycoprotein.

“VSV-G” refers to a type III viral fusion protein that mediates fusion between the viral envelope of VSV and a host cell membrane to release the viral genome into the host cell.

“Replication incompetent VSV” refers to VSV in which the gene encoding the G envelope glycoprotein is deleted or mutated to produce a nonfunctional protein.

As used herein, the terms “vector” or “plasmid” refer to a polynucleotide construct designed for transduction/transfection of one or more cell types. Vectors or plasmids may be, for example, “expression vectors” or “expression plasmids” which are designed for expression of a nucleotide sequence in a host cell (e.g., a packaging cell).

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The term “heterologous” refers to a combination of elements not naturally occurring in a virus or cell. For example, heterologous DNA refers to the DNA not naturally located in the cell, or in a chromosomal site of the cell. The heterologous DNA may include a gene foreign to the cell. In the context of VSV, a “heterologous glycoprotein” refers to a glycoprotein that is not present in the envelope of wild-type VSV.

The terms “cell”, “host cell”, “cell culture” and the like as used herein are intended to include any individual cell or cell culture which can be or have been recipients for viruses, vectors or the incorporation of exogenous nucleic acid molecules, polynucleotides and/or proteins. It is also intended to include progeny of a single cell. The cells are preferably eukaryotic, but may be prokaryotic and include, but are not limited to, bacterial cells, yeast cells, animal cells, and mammalian cells (e.g., murine, rat, simian or human).

“Expression” includes transcription and/or translation.

“Encoded by” or “encoding” refers to a nucleic acid sequence which codes for a polypeptide sequence (i.e., amino acid sequence) of a polypeptide encoded by the nucleic acid sequence.

“Replication” and “propagation” are used interchangeably and refer to the ability of a virus of the invention to reproduce or proliferate. These terms are well understood in the art. For purposes of this invention, replication involves production of VSV proteins and is generally directed to reproduction of VSV. Replication can be measured using assays standard in the art. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

The term “multiplicity of infection” or “MOI” refers to the average number of viral particles that infects a single cell. The “MOI” is calculated by dividing the total number of viral plaque forming units (PFU) with the total number of cells being infected.

The term “plaque” or “viral plaque” refers to a clear, often round patch of lysed cells in an otherwise opaque layer of a cell culture. A “plaque-forming unit” or “PFU” refers to the average number of infectious viral particles per unit volume. For example, if a virus solution has 100 PFU/ml, this means that every one milliliter of this virus solution has 100 virus particles that can each form a plaque. PFU/mL is the conventional means to refer to a concentration of a plaque forming virus preparation. However, PFU is generally used interchangeably with “infectious unit” or “IU” and represents units of infectious virus in a virus preparation.

The terms “culture fluid”, “cell culture fluid”, “cell culture media”, “cell culture medium”, “media” and/or “bioreactor fluid” are used interchangeably, and refer to the media or solution in which the cell culture is grown.

A cell suspension, suspension culture, or suspension cell culture is a type of cell culture in which single cells or small aggregates of cells are allowed to function and multiply in an agitated growth medium, thus forming a suspension. Suspension cell cultures are contrasted with adherent cell cultures in which the cells multiply while anchored to a physical substrate.

The term “growing” or “growth” as used herein refer to the in vitro propagation of virus in cells of various kinds. The growing/growth of virus in cells in the laboratory involves inoculating the cells with the virus, followed by incubating to allow virus production and then harvesting the cell culture medium containing the virus. Virus-infected cells are normally grown in a growth medium within culture vessels (such as flasks or bioreactors) and the cultures are maintained in cell incubators with specified temperature, humidity and gas composition. However, culture conditions can vary depending on the cell type and can be altered to induce changes in the cells or to support or enhance virus production by the cells.

The term “harvesting”, as used herein, refers to the collection of cells or cell culture medium in preparation for isolation and purification of virus following the infection of a cell or cell line with any of the virus strains or serotypes described herein.

The terms “isolated,” “isolating,” “purified,” or “purifying” mean that the material is removed from the production environment. Thus, a virus that is isolated or purified, or the process of doing so, refers to removal of the virus from the cell culture and packaging cells in which the virus was produced.

A “gene” as used in the context of the present invention is a sequence of nucleotides in a nucleic acid molecule (chromosome, plasmid, etc.) with which a genetic function is associated. A gene can encode an expressed product, such as a polypeptide or a polynucleotide (e.g., tRNA). Typically, a gene includes coding sequences, such as polypeptide encoding sequences, and non-coding sequences, such as introns or regulatory sequences.

The terms “genetically modified” or “recombinant” generally refer to the introduction of one or more mutations or deletions in the viral genome by any means known to those skilled in the art.

The terms “protein”, “polypeptide” and “peptide” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The term “amino acid” refers to either natural and/or unnatural or synthetic amino acids, including both the D or L optical isomers, and amino acid analogs. The one letter and three letter codes for each of the natural amino acids are known to those skilled in the art.

As used herein, the term “comprising” and its cognates are used in their inclusive sense; that is, equivalent to the term “including” and its corresponding cognates.

### General Description

The present disclosure provides methods for the production of recombinant vesicular stomatitis virus (rVSV) in a scalable suspension cell culture production system that can yield viral titers comparable to adherent cell culture processes. In particular, the methods of the present disclosure include specific combinations of culture, infection and harvesting parameters to surprising produce high titers of replication-incompetent rVSV in suspension cell culture. The methods can be used, for example, to produce replication-incompetent rVSV with envelope glycoproteins of any one of a variety of enveloped viruses (e.g., SARS-CoV-2 or Ebola) for use as a pseudovirus in, e.g., viral antibody neutralization assays, or for use in vector-based vaccines.

### Methods for Producing Replication-Incompetent Recombinant VSV

Aspects of the disclosure are directed to methods of producing replication-incompetent recombinant VSV particles that incorporate an envelope glycoprotein expressed from a plasmid or other vector in a packaging cell from which the rVSV particles bud.

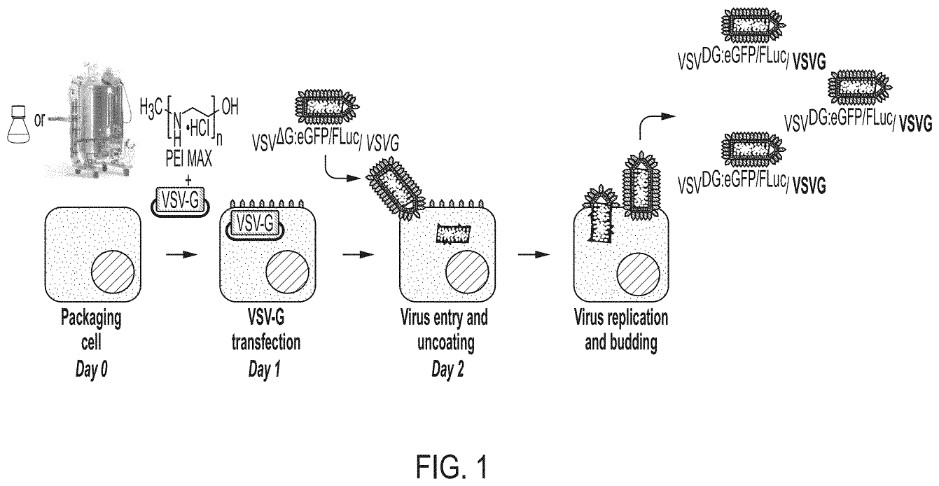

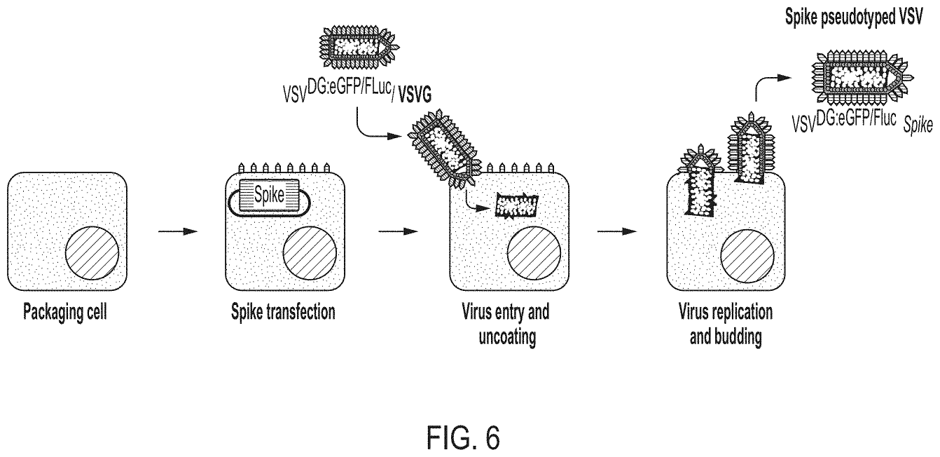

With reference to the exemplary production schematic illustrated in FIG. 1, a method of producing replication-incompetent recombinant vesicular stomatitis virus (rVSV), may comprise: (a) inoculating a suspension cell culture medium (in, e.g., a bioreactor) with a plurality of packaging cells (e.g., HEK293 cells) at Day 0; (b) transfecting the packaging cells in the suspension cell culture medium with a plasmid comprising a nucleic acid molecule encoding a viral envelope glycoprotein to produce a population of packaging cells expressing the viral envelope glycoprotein at Day 1 (VSV-G is illustrated in FIG. 1, but this could be substituted with any viral envelope glycoprotein, such as a spike glycoprotein as illustrated in FIG. 6); (c) introducing rVSV into the suspension cell culture medium at a multiplicity of infection (MOI) rate of from 0.001 to 3 to infect the population of packaging cells expressing the viral envelope glycoprotein at Day 2, wherein the rVSV has been engineered such that the rVSV does not express a functional envelope glycoprotein (e.g., rVSV-ΔG); and (d) isolating rVSV produced from the population of packaging cells from 15 to 65 hours post infection following replication and budding of the virus, wherein the viral envelope glycoprotein in incorporated into the viral envelope of the isolated rVSV (i.e., when the virus buds through the membrane of the packaging cell).

The packaging cell (or host cell) used for recombinant VSV production can be any cell in which VSV replicates, e.g., mammalian cells and some insect (e.g., Drosophila) cells. Immortilized or tumor cell lines can be used. A number of cell lines commonly known in the art are available for use. By way of example, such cell lines include, but are not limited to, BHK (baby hamster kidney) cells, CHO (Chinese hamster ovary) cells, HeLA (human) cells, mouse L cells, Vero (monkey) cells, African green monkey kidney (AGMK) cells, ESK-4, PK-15, EMSK cells, MDCK (Madin-Darby canine kidney) cells, MDBK (Madin-Darby bovine kidney) cells, HEK293 (human) cells, and Hep-2 cells. Such cell lines are publicly available, for example, from the ATCC and other culture depositories. In some embodiments, the packaging or host cells are mammalian cells. In some cases, the packaging cells or host cells are primate cells or human cells. In some embodiments, the packaging cells or host cells are human embryonic kidney (HEK) cells. In some instances, the HEK cells are HEK293 cells, and may be selected from HEK293F cells, HEK293T cells, HEK293SF cells, and HEK293S cells. In some embodiments, the packaging cells or host cells are HEK293F cells. In some cases, the packaging or host cells are HEK293F cells that grow in suspension cell culture.

The culture medium (or production medium) used for suspension cell culture of the packaging or host cells can be any suitable product. Preferably, the culture medium is animal-derived component free, protein free, and chemically defined. An exemplary culture medium is CTS™ LV-MAX™ production medium (ThermoFisher Scientific).

Transfection of the packaging cells or host cells can be accomplished by any suitable method known in the art. In some embodiments, transfection is accomplished with use of a transfection reagent (to facilitate uptake of the vector or plasmid), such as a polyethylenimine transfection reagent (e.g., PEI MAX™; Polysciences), a lipofectamine transfection reagent, or a FectoVIR®-AAV transfection reagent (Polyplus, France). Alternatively, the plasmid DNA uptake can also be enhanced by electroporation of the cells, whereby a high voltage current is applied across cuvette containing cells and DNA for milliseconds.

In various embodiments, transfection of the vector or plasmid into the plurality of packaging or host cells is performed when the cell density of the packaging cells reaches a sufficient density. In some embodiments, transfection is undertaken when the cell density reaches at least 3×10⁶ packaging cells/mL. In various embodiments, the cell density is at least about 1×10⁶ cells/mL, at least about 1.5×10⁶ cells/mL, at least about 2×10⁶ cells/mL, at least about 2.5×10⁶ cells/mL, at least about 3×10⁶ cells/mL, at least about 3.5×10⁶ cells/mL, at least about 4×10⁶ cells/mL, at least about 4.5×10⁶ cells/mL, or at least about 5×10⁶ cells/mL.

In some cases, transfection of the packaging cells or host cells is accomplished using a DNA concentration of at least 1.5 μg/mL. In various embodiments, the DNA concentration included in the vector or plasmid is at least about 0.5 μg/mL, at least about 0.6 μg/mL, at least about 0.7 μg/mL, at least about 0.8 μg/mL, at least about 0.9 μg/mL, at least about 1.0 μg/mL, at least about 1.1 μg/mL, at least about 1.2 μg/mL, at least about 1.3 μg/mL, at least about 1.4 μg/mL, at least about 1.5 μg/mL, at least about 1.6 μg/mL, at least about 1.7 μg/mL, at least about 1.8 μg/mL, at least about 1.9 μg/mL, at least about 2.0 μg/mL, at least about 2.5 μg/mL, at least about 3.0 μg/mL, at least about 3.5 μg/mL, at least about 4.0 μg/mL, at least about 5.5 μg/mL, or at least about 5.0 μg/mL.

In various embodiments, transfection is accomplished via use of a transfection reagent:DNA ratio of from about 1 to about 5. In some cases, the transfection reagent:DNA ratio is from about 2 to about 3. In some cases, the transfection reagent:DNA ratio is 2.5. In various embodiments, the transfection reagent:DNA ratio is about 1, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0.

In various embodiments, the vector or plasmid transfected into the packaging cells (or host cells) comprises a nucleic acid molecule encoding a viral envelope glycoprotein that may be a heterologous glycoprotein (i.e., a glycoprotein not normally produced by VSV). In some cases, the viral envelope glycoprotein is a vesicular stomatitis virus G protein. In some cases, the viral envelope glycoprotein is a class I fusion protein. In some cases, the viral envelope glycoprotein is a class II fusion protein. In some cases, the viral envelope glycoprotein is a class III fusion protein. In some embodiments, the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses.

In various embodiments, the vector or plasmid transfected into the packaging cells (or host cells) comprises a nucleic acid molecule encoding a coronavirus spike glycoprotein, e.g. a spike protein from SARS-CoV-2. In some cases, the spike glycoprotein comprises the amino acid sequence of SEQ ID NO: 1. In some cases, the spike glycoprotein comprises the amino acid sequence of SEQ ID NO: 2.

In various embodiments, the vector or plasmid transfected into the packaging cells (or host cells) comprises a nucleic acid molecule encoding an ebola virus glycoprotein. In some cases, the ebola virus glycoprotein comprises the amino acid sequence of SEQ ID NO: 3. In some cases, the ebola virus glycoprotein comprises the amino acid sequence of SEQ ID NO: 4.

In any of the various embodiments of the methods discussed herein, the suspension cell culture medium is not exchanged prior to introducing rVSV into the suspension cell culture medium (discussed below). Although media exchange prior to or near the time of infection of the packaging cells with the virus is often regarded as being advantageous to remove residual elements from the transfection process, the present inventors have surprising discovered that these advantages are diminished in the context of other factors (e.g., MOI and harvest timing) such that media exchange can be avoided, thereby enhancing the scalability of the production process.

In various embodiments, infection of the packaging cells (or host cells) with the recombinant VSV (not expressing a functional envelope glycoprotein) is performed by introducing the virus in to the suspension cell culture medium at a multiplicity of infection (MOI) rate of from about 0.001 to about 5. In some cases, the MOI is from about 0.001 to about 0.5. In some cases, the MOI is from about 0.001 to about 1. In some cases, the MOI is from about 0.005 to about 3. In some cases, the MOI is from about 0.005 to about 1.5. In some cases, the MOI is from about 0.001 to about 0.1. In some cases, the MOI is from about 0.005 to about 0.05. In some cases, the MOI is from about 0.008 to about 0.012. In some cases, the MOI is about 0.01±0.001. In some cases, the MOI is from about 0.5 to about 1.5. In some cases, the MOI is from about 0.8 to about 1.2. In some cases, the MOI is about 1±0.1. In various embodiments, the MOI is about 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.011, 0.012, 0.013, 0.014, 0.015, 0.016, 0.017, 0.018, 0.019, 0.02, 0.0225, 0.025, 0.0275, 0.03, 0.0325, 0.035, 0.0375, 0.04, 0.0425, 0.045, 0.0475, or 0.5. In some cases, the MOI is about 0.1, 0.15, 0.2, 0.25, 0.3, 0.35, 0.4, 0.05, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, or 5.

In the methods discussed herein, the suspension cell culture medium is maintained at a temperature during inoculation and transfection, and the temperature of the suspension cell culture medium is reduced to a lower temperature when infecting the packaging cells with the rVSV. In some cases, the temperature of the suspension cell culture is at about 37° C. during inoculation and transfection, and is reduced to a temperature of from about 32° C. to about 36.5° C. at or near (e.g., within 1 hour) the time of infection. In some cases, the temperature is reduced to about 35.5° C. In some cases, the temperature is reduced to about 34° C. In various embodiments, the temperature of the cell culture during inoculation and transfection is 36.6° C., 36.7° C., 36.8° C., 36.9° C., 37° C., 37.1° C., 37.2° C., 37.3° C., 37.4° C., 37.5° C., 37.6° C., 37.7° C., 37.8° C., 37.9° C., or 38° C. In various embodiments, including those mentioned in the immediately preceding sentence, the reduced temperature is 32° C., 32.1° C., 32.2° C., 32.3° C., 32.4° C., 32.5° C., 32.6° C., 32.7° C., 32.8° C., 32.9° C., 33° C., 33.1° C., 33.2° C., 33.3° C., 33.4° C., 33.5° C., 33.6° C., 33.7° C., 33.8° C., 33.9° C., 34° C., 34.1° C., 34.2° C., 34.3° C., 34.4° C., 34.5° C., 34.6° C., 34.7° C., 34.8° C., 34.9° C., 35° C., 35.1° C., 35.2° C., 35.3° C., 35.4° C., 35.5° C., 35.6° C., 35.7° C., 35.8° C., 35.9° C., 36° C., 36.1° C., 36.2° C., 36.3° C., 36.4° C., or 36.5° C.

In various embodiments, the infected packaging cells are culture for a period of time before the viral particles are harvested and isolated. In some embodiments, the rVSV particles produced from the packaging cells are isolated from about 40 to about 50 hours post infection (hpi). In some cases, the rVSV particles produced from the packaging cells are isolated from about 43 hpi to about 47 hpi. In various embodiments, the rVSV particles produced from the packaging cells are isolated about 44 hpi or about 45 hpi. In some cases, the rVSV particles produced from the packaging cells are isolated 40 hpi, 40.1 hpi, 40.2 hpi, 40.3 hpi, 40.4 hpi, 40.5 hpi, 40.6 hpi, 40.7 hpi, 40.8 hpi, 40.9 hpi, 41 hpi, 41.1 hpi, 41.2 hpi, 41.3 hpi, 41.4 hpi, 41.5 hpi, 41.6 hpi, 41.7 hpi, 41.8 hpi, 41.9 hpi 42 hpi, 42.1 hpi, 42.2 hpi, 42.3 hpi, 42.4 hpi, 42.5 hpi, 42.6 hpi, 42.7 hpi, 42.8 hpi, 42.9 hpi 43 hpi, 43.1 hpi, 43.2 hpi, 43.3 hpi, 43.4 hpi, 43.5 hpi, 43.6 hpi, 43.7 hpi, 43.8 hpi, 43.9 hpi 44 hpi, 44.1 hpi, 44.2 hpi, 44.3 hpi, 44.4 hpi, 44.5 hpi, 44.6 hpi, 44.7 hpi, 44.8 hpi, 44.9 hpi 45 hpi, 45.1 hpi, 45.2 hpi, 45.3 hpi, 45.4 hpi, 45.5 hpi, 45.6 hpi, 45.7 hpi, 45.8 hpi, 45.9 hpi 46 hpi, 46.1 hpi, 46.2 hpi, 46.3 hpi, 46.4 hpi, 46.5 hpi, 46.6 hpi, 46.7 hpi, 46.8 hpi, 46.9 hpi 47 hpi, 47.1 hpi, 47.2 hpi, 47.3 hpi, 47.4 hpi, 47.5 hpi, 47.6 hpi, 47.7 hpi, 47.8 hpi, 47.9 hpi 48 hpi, 48.1 hpi, 48.2 hpi, 48.3 hpi, 48.4 hpi, 48.5 hpi, 48.6 hpi, 48.7 hpi, 48.8 hpi, 48.9 hpi 49 hpi, 49.1 hpi, 49.2 hpi, 49.3 hpi, 49.4 hpi, 49.5 hpi, 49.6 hpi, 49.7 hpi, 49.8 hpi, 49.9 hpi, or 50 hpi, In some cases, the rVSV particles produced from the packaging cells are isolated from about 15 hpi to about 35 hpi. In some cases, the rVSV particles produced from the packaging cells are isolated from about 20 hpi to about 30 hpi. In various embodiments, the rVSV particles produced from the packaging cells are isolated about 24 hpi or about 25 hpi. In some cases, the rVSV particles produced from the packaging cells are isolated 20 hpi, 20.1 hpi, 20.2 hpi, 20.3 hpi, 20.4 hpi, 20.5 hpi, 20.6 hpi, 20.7 hpi, 20.8 hpi, 20.9 hpi, 21 hpi, 21.1 hpi, 21.2 hpi, 21.3 hpi, 21.4 hpi, 21.5 hpi, 21.6 hpi, 21.7 hpi, 21.8 hpi, 21.9 hpi 22 hpi, 22.1 hpi, 22.2 hpi, 22.3 hpi, 22.4 hpi, 22.5 hpi, 22.6 hpi, 22.7 hpi, 22.8 hpi, 22.9 hpi 23 hpi, 23.1 hpi, 23.2 hpi, 23.3 hpi, 23.4 hpi, 23.5 hpi, 23.6 hpi, 23.7 hpi, 23.8 hpi, 23.9 hpi 24 hpi, 24.1 hpi, 24.2 hpi, 24.3 hpi, 24.4 hpi, 24.5 hpi, 24.6 hpi, 24.7 hpi, 24.8 hpi, 24.9 hpi 25 hpi, 25.1 hpi, 25.2 hpi, 25.3 hpi, 25.4 hpi, 25.5 hpi, 25.6 hpi, 25.7 hpi, 25.8 hpi, 25.9 hpi 26 hpi, 26.1 hpi, 26.2 hpi, 26.3 hpi, 26.4 hpi, 26.5 hpi, 26.6 hpi, 26.7 hpi, 26.8 hpi, 26.9 hpi 27 hpi, 27.1 hpi, 27.2 hpi, 27.3 hpi, 27.4 hpi, 27.5 hpi, 27.6 hpi, 27.7 hpi, 27.8 hpi, 27.9 hpi 28 hpi, 28.1 hpi, 28.2 hpi, 28.3 hpi, 28.4 hpi, 28.5 hpi, 28.6 hpi, 28.7 hpi, 28.8 hpi, 28.9 hpi 29 hpi, 29.1 hpi, 29.2 hpi, 29.3 hpi, 29.4 hpi, 29.5 hpi, 29.6 hpi, 29.7 hpi, 29.8 hpi, 29.9 hpi, or 30 hpi, In some cases, the rVSV particles produced from the packaging cells are isolated from about 30 hpi to about 40 hpi. In some cases, the rVSV particles produced from the packaging cells are isolated 30 hpi, 30.1 hpi, 30.2 hpi, 30.3 hpi, 30.4 hpi, 30.5 hpi, 30.6 hpi, 30.7 hpi, 30.8 hpi, 30.9 hpi, 31 hpi, 31.1 hpi, 31.2 hpi, 31.3 hpi, 31.4 hpi, 31.5 hpi, 31.6 hpi, 31.7 hpi, 31.8 hpi, 31.9 hpi 32 hpi, 32.1 hpi, 32.2 hpi, 32.3 hpi, 32.4 hpi, 32.5 hpi, 32.6 hpi, 32.7 hpi, 32.8 hpi, 32.9 hpi 33 hpi, 33.1 hpi, 33.2 hpi, 33.3 hpi, 33.4 hpi, 33.5 hpi, 33.6 hpi, 33.7 hpi, 33.8 hpi, 33.9 hpi 34 hpi, 34.1 hpi, 34.2 hpi, 34.3 hpi, 34.4 hpi, 34.5 hpi, 34.6 hpi, 34.7 hpi, 34.8 hpi, 34.9 hpi 35 hpi, 35.1 hpi, 35.2 hpi, 35.3 hpi, 35.4 hpi, 35.5 hpi, 35.6 hpi, 35.7 hpi, 35.8 hpi, 35.9 hpi 36 hpi, 36.1 hpi, 36.2 hpi, 36.3 hpi, 36.4 hpi, 36.5 hpi, 36.6 hpi, 36.7 hpi, 36.8 hpi, 36.9 hpi 37 hpi, 37.1 hpi, 37.2 hpi, 37.3 hpi, 37.4 hpi, 37.5 hpi, 37.6 hpi, 37.7 hpi, 37.8 hpi, 37.9 hpi 38 hpi, 38.1 hpi, 38.2 hpi, 38.3 hpi, 38.4 hpi, 38.5 hpi, 38.6 hpi, 38.7 hpi, 38.8 hpi, 38.9 hpi 39 hpi, 39.1 hpi, 39.2 hpi, 39.3 hpi, 39.4 hpi, 39.5 hpi, 39.6 hpi, 39.7 hpi, 39.8 hpi, 39.9 hpi, or 40 hpi,

VSV is generally secreted from the host or packaging cell into the culture media. Thus, isolating the VSV product may include collection from the cell culture fluid. In some cases, rVSV produced by cell lines can be isolated using for example, an affinity matrix. Briefly, methods for isolating a rVSV may comprise adding the VSV to an affinity matrix, to produce bound VSV, washing the bound VSV, and eluting the VSV from the affinity matrix. The present disclosure encompasses a modified VSV that comprises a non-naturally occurring fusion protein on the outer surface of the virus. The non-native protein may be a fusion protein comprising an affinity tag and a viral envelope glycoprotein or it may be derived from a packaging cell. Packaging cell lines may be engineered to express one or more affinity tags on their plasma membranes which would be acquired by the virus as it buds through the membrane. One example of an affinity tag is the use of Histidine residues which bind to immobilized nickel columns. Affinity tags also include antibodies. Other protocols for affinity purification may be used as known within the art, for example, but not limited to, batch processing, a solution of virus and affinity matrix, pelleting the VSV-bound matrix by centrifugation, and isolating the virus. Alternatively, VSV can be collected and purified from culture supernatants, and the supernatants clarified to remove cellular debris. One method of isolating and concentrating the virus is by passage of the supernatant through a tangential flow membrane concentration. The harvest can be further reduced in volume by pelleting through a glycerol cushion and by concentration on a cesium chloride or sucrose step gradient or other form of gradient.

In various embodiments, the suspension cell culture production methods of the present disclosure yield rVSV at an infectious titer of at least 1×10⁶ plaque forming units (pfu)/mL. In some cases, the suspension cell culture production methods of the present disclosure yield rVSV at an infectious titer of at least 1×10⁷ pfu/mL. In some cases, the suspension cell culture production methods of the present disclosure yield rVSV at an infectious titer of at least 1×10⁸ pfu/mL. In some cases, the suspension cell culture production methods of the present disclosure yield rVSV at an infectious titer of at least 5×10⁸ pfu/mL. In some cases, the suspension cell culture production methods of the present disclosure yield rVSV at an infectious titer of at least 1×10⁹ pfu/mL. In various embodiments, the suspension cell culture production methods of the present disclosure yield rVSV at an infectious titer of, or at least of, 1×10⁶ pfu/mL, 2×10⁶ pfu/mL, 3×10⁶ pfu/mL, 4×10⁶ pfu/mL, 5×10⁶ pfu/mL, 6×10⁶ pfu/mL, 7×10⁶ pfu/mL, 8×10⁶ pfu/mL, 9×10⁶ pfu/mL, 1×10⁷ pfu/mL, 2×10⁷ pfu/mL, 3×10⁷ pfu/mL, 4×10⁷ pfu/mL, 5×10⁷ pfu/mL, 6×10⁷ pfu/mL, 7×10⁷ pfu/mL, 8×10⁷ pfu/mL, 9×10⁷ pfu/mL, 1×10⁸ pfu/mL, 2×10⁸ pfu/mL, 3×10⁸ pfu/mL, 4×10⁸ pfu/mL, 5×10⁸ pfu/mL, 6×10⁸ pfu/mL, 7×10⁸ pfu/mL, 8×10⁸ pfu/mL, 9×10⁸ pfu/mL, 1×10⁹ pfu/mL, 2×10⁹ pfu/mL, 3×10⁹ pfu/mL, 4×10⁹ pfu/mL, 5×10⁹ pfu/mL, 6×10⁹ pfu/mL, 7×10⁹ pfu/mL, 8×10⁹ pfu/mL, or 9×10⁹ pfu/mL.

In various embodiments, the suspension cell culture production methods of the present disclosure yield rVSV at an infectious titer that is at least comparable to the titer produced by an adherent cell culture control (which produces the same rVSV). In some cases, the suspension cell culture production methods of the present disclosure yield rVSV at an infectious titer that is at least 1.5 fold, at least 2 fold, at least 2.5 fold, or at least 3 fold greater than the titer produced by an adherent cell culture control (which produces the same rVSV).

### Vesicular Stomatitis Virus (VSV)

VSV, a member of the Rhabdoviridae family, is a negative-stranded virus that replicates in the cytoplasm of infected cells, does not undergo genetic recombination or reassortment, has no known transforming potential and does not integrate any part of it genome into the host. VSV comprises an about 11 kilobase genome that encodes for five proteins referred to as the nucleocapsid (N), polymerase proteins (L) and (P), surface glycoprotein (G) and a peripheral matrix protein (M). The genome is tightly encased in nucleocapsid (N) protein and also comprises the polymerase proteins (L) and (P). Following infection of the cell, the polymerase proteins initiate the transcription of five subgenomic viral mRNAs, from the negative-sense genome, that encode the viral proteins. The polymerase proteins are also responsible for the replication of the full-length viral genomes that are packaged into progeny virions. The matrix (M) protein binds to the RNA genome/nucleocapsid core (RNP) and also to the glycosylated (G) protein, which extends from the outer surface in an array of spike like projections and is responsible for binding to cell surface receptors and initiating the infectious process. Recombinant VSV, as discussed herein, may be identical to the VSV discussed above, except for deletion of the gene encoding the glycosylated (G) protein, or mutation of the gene encoding the glycosylated (G) protein such that a nonfunctional protein is produced. The recombinant VSV used for infection, although not encoding a functional G glycoprotein, includes the G glycoprotein in its viral envelope.

Following attachment of VSV through the (G) protein to receptor(s) on the host surface, the virus penetrates the host and uncoats to release the RNP particles. The polymerase proteins, which are carried in with the virus, bind to the 3′ end of the genome and sequentially synthesize the individual mRNAs encoding N, P, M, and L, followed by negative-sense progeny genomes. Newly synthesized N, P and L proteins associate in the cytoplasm and form RNP cores which bind to regions of the plasma membrane rich in both M protein, and the glycoprotein encoded by the transfected plasmid. Viral particles form and budding or release of progeny virus ensues.

Any of various VSV strains can be utilized in the methods of the present disclosure. VSV strains include Indiana, New Jersey, Piry, Colorado, Coccal, Chandipura and San Juan. The complete nucleotide and deduced protein sequence of a VSV genome is known and is available as Genbank VSVCG, accession number J02428; NCBI Seq ID 335873; and is published in Rose and Schubert, 1987, in The Viruses: The Rhabdoviruses, Plenum Press, NY. pp. 129-166. VSV New Jersey strain is available from the American Type Culture Collection (ATCC) and has ATCC accession number VR-159. VSV Indiana strain is available from the ATCC and has ATCC accession number VR-1421.

### Exemplary Production Methods

In one embodiment, the present disclosure provides a method of producing replication-incompetent recombinant vesicular stomatitis virus (rVSV), comprising: (a) inoculating a suspension cell culture medium with a plurality of HEK293 cells selected from HEK293F cells, HEK293T cells, HEK293SF cells, or HEK293S cells, wherein the suspension cell culture medium is at a temperature of from about 36.6° C. to about 37.4° C.; (b) transfecting the HEK293 cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein; (c) reducing the temperature of the suspension cell culture medium to from about 32.5° C. to about 35.5° C., and introducing rVSV into the suspension cell culture medium at a multiplicity of infection (MOI) rate of from about 0.005 to about 0.015 to infect the population of HEK293 cells expressing the viral envelope glycoprotein, wherein the rVSV has been engineered such that the rVSV does not express a functional envelope glycoprotein; and (d) isolating rVSV produced from the population of packaging cells from 40 to 48 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293 cells selected from HEK293F cells, HEK293T cells, HEK293SF cells, or HEK293S cells, wherein the suspension cell culture medium is at a temperature of from about 36.8° C. to about 37.2° C.; (b) transfecting the HEK293 cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein; (c) reducing the temperature of the suspension cell culture medium to from about 33° C. to about 35° C., and introducing rVSV into the suspension cell culture medium at a multiplicity of infection (MOI) rate of from about 0.008 to about 0.012 to infect the population of HEK293 cells expressing the viral envelope glycoprotein, wherein the rVSV has been engineered such that the rVSV does not express a functional envelope glycoprotein; and (d) isolating rVSV produced from the population of packaging cells from 42 to 46 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293 cells selected from HEK293F cells, HEK293T cells, HEK293SF cells, or HEK293S cells, wherein the suspension cell culture medium is at a temperature of 37° C.±0.1° C.; (b) transfecting the HEK293 cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein; (c) reducing the temperature of the suspension cell culture medium to about 34° C.±0.1° C. and introducing rVSV into the suspension cell culture medium at a multiplicity of infection (MOI) rate of about 0.01 to infect the population of HEK293 cells expressing the viral envelope glycoprotein, wherein the rVSV has been engineered such that the rVSV does not express a functional envelope glycoprotein; and (d) isolating rVSV produced from the population of packaging cells from 43 to 45 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.6° C. to about 37.4° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses; (c) reducing the temperature of the suspension cell culture medium to from about 32.5° C. to about 35.5° C., and introducing rVSV into the suspension cell culture medium at a multiplicity of infection (MOI) rate of from about 0.005 to about 0.015 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 40 to 48 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.8° C. to about 37.2° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses; (c) reducing the temperature of the suspension cell culture medium to from about 33° C. to about 35° C., and introducing rVSV into the suspension cell culture medium at a multiplicity of infection (MOI) rate of from about 0.008 to about 0.012 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 42 to 46 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of 37° C.±0.1° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses; (c) reducing the temperature of the suspension cell culture medium to about 34° C.±0.1° C. and introducing rVSV into the suspension cell culture medium at a multiplicity of infection (MOI) rate of about 0.01 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 43 to 45 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.6° C. to about 37.4° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a coronavirus spike protein (e.g., the spike protein of SARS-CoV-2), optionally comprising the amino acid sequence of SEQ ID NO: 1 or SEQ ID NO: 2; (c) reducing the temperature of the suspension cell culture medium to from about 32.5° C. to about 35.5° C., and introducing rVSV into the suspension cell culture medium at a multiplicity of infection (MOI) rate of from about 0.005 to about 0.015 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 40 to 48 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.8° C. to about 37.2° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a coronavirus spike protein (e.g., the spike protein of SARS-CoV-2), optionally comprising the amino acid sequence of SEQ ID NO: 1 or SEQ ID NO: 2; (c) reducing the temperature of the suspension cell culture medium to from about 33° C. to about 35° C., and introducing rVSV into the suspension cell culture medium at a multiplicity of infection (MOI) rate of from about 0.008 to about 0.012 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 42 to 46 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of 37° C.±0.1° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a coronavirus spike protein (e.g., the spike protein of SARS-CoV-2), optionally comprising the amino acid sequence of SEQ ID NO: 1 or SEQ ID NO: 2; (c) reducing the temperature of the suspension cell culture medium to about 34° C.±0.1° C. and introducing rVSV into the suspension cell culture medium at a multiplicity of infection (MOI) rate of about 0.01 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 43 to 45 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.6° C. to about 37.4° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is an ebola virus glycoprotein, optionally comprising the amino sequence of SEQ ID NO: 3 or SEQ ID NO: 4; (c) reducing the temperature of the suspension cell culture medium to from about 32.5° C. to about 35.5° C., and introducing rVSV into the suspension cell culture medium at a multiplicity of infection (MOI) rate of from about 0.005 to about 0.015 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 40 to 48 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.8° C. to about 37.2° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is an ebola virus glycoprotein, optionally comprising the amino sequence of SEQ ID NO: 3 or SEQ ID NO: 4; (c) reducing the temperature of the suspension cell culture medium to from about 33° C. to about 35° C., and introducing rVSV into the suspension cell culture medium at a multiplicity of infection (MOI) rate of from about 0.008 to about 0.012 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 42 to 46 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of 37° C.±0.1° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is an ebola virus glycoprotein, optionally comprising the amino sequence of SEQ ID NO: 3 or SEQ ID NO: 4; (c) reducing the temperature of the suspension cell culture medium to about 34° C.±0.1° C. and introducing rVSV into the suspension cell culture medium at a multiplicity of infection (MOI) rate of about 0.01 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 43 to 45 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293 cells selected from HEK293F cells, HEK293T cells, HEK293SF cells, or HEK293S cells, wherein the suspension cell culture medium is at a temperature of from about 36.6° C. to about 37.4° C.; (b) transfecting the HEK293 cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein; (c) reducing the temperature of the suspension cell culture medium to from about 32.5° C. to about 35.5° C., and introducing rVSV into the suspension cell culture medium at a multiplicity of infection (MOI) rate of from about 0.5 to about 1.5 to infect the population of HEK293 cells expressing the viral envelope glycoprotein, wherein the rVSV has been engineered such that the rVSV does not express a functional envelope glycoprotein; and (d) isolating rVSV produced from the population of packaging cells from 20 to 28 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293 cells selected from HEK293F cells, HEK293T cells, HEK293SF cells, or HEK293S cells, wherein the suspension cell culture medium is at a temperature of from about 36.8° C. to about 37.2° C.; (b) transfecting the HEK293 cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein; (c) reducing the temperature of the suspension cell culture medium to from about 33° C. to about 35° C., and introducing rVSV into the suspension cell culture medium at a multiplicity of infection (MOI) rate of from about 0.8 to about 1.2 to infect the population of HEK293 cells expressing the viral envelope glycoprotein, wherein the rVSV has been engineered such that the rVSV does not express a functional envelope glycoprotein; and (d) isolating rVSV produced from the population of packaging cells from 22 to 26 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293 cells selected from HEK293F cells, HEK293T cells, HEK293SF cells, or HEK293S cells, wherein the suspension cell culture medium is at a temperature of 37° C.±0.1° C.; (b) transfecting the HEK293 cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein; (c) reducing the temperature of the suspension cell culture medium to about 34° C.±0.1° C. and introducing rVSV into the suspension cell culture medium at a multiplicity of infection (MOI) rate of about 1 to infect the population of HEK293 cells expressing the viral envelope glycoprotein, wherein the rVSV has been engineered such that the rVSV does not express a functional envelope glycoprotein; and (d) isolating rVSV produced from the population of packaging cells from 23 to 25 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.6° C. to about 37.4° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses; (c) reducing the temperature of the suspension cell culture medium to from about 32.5° C. to about 35.5° C., and introducing rVSV into the suspension cell culture medium at a multiplicity of infection (MOI) rate of from about 0.5 to about 1.5 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 20 to 28 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.8° C. to about 37.2° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses; (c) reducing the temperature of the suspension cell culture medium to from about 33° C. to about 35° C., and introducing rVSV into the suspension cell culture medium at a multiplicity of infection (MOI) rate of from about 0.8 to about 1.2 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 22 to 26 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of 37° C.±0.1° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses; (c) reducing the temperature of the suspension cell culture medium to about 34° C.±0.1° C. and introducing rVSV into the suspension cell culture medium at a multiplicity of infection (MOI) rate of about 1 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 23 to 25 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.6° C. to about 37.4° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a coronavirus spike protein (e.g., the spike protein of SARS-CoV-2), optionally comprising the amino acid sequence of SEQ ID NO: 1 or SEQ ID NO: 2; (c) reducing the temperature of the suspension cell culture medium to from about 32.5° C. to about 35.5° C., and introducing rVSV into the suspension cell culture medium at a multiplicity of infection (MOI) rate of from about 0.5 to about 1.5 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 20 to 28 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.8° C. to about 37.2° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a coronavirus spike protein (e.g., the spike protein of SARS-CoV-2), optionally comprising the amino acid sequence of SEQ ID NO: 1 or SEQ ID NO: 2; (c) reducing the temperature of the suspension cell culture medium to from about 33° C. to about 35° C., and introducing rVSV into the suspension cell culture medium at a multiplicity of infection (MOI) rate of from about 0.8 to about 1.2 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 22 to 26 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of 37° C.±0.1° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is a coronavirus spike protein (e.g., the spike protein of SARS-CoV-2), optionally comprising the amino acid sequence of SEQ ID NO: 1 or SEQ ID NO: 2; (c) reducing the temperature of the suspension cell culture medium to about 34° C.±0.1° C. and introducing rVSV into the suspension cell culture medium at a multiplicity of infection (MOI) rate of about 1 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 23 to 25 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.6° C. to about 37.4° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is an ebola virus glycoprotein, optionally comprising the amino sequence of SEQ ID NO: 3 or SEQ ID NO: 4; (c) reducing the temperature of the suspension cell culture medium to from about 32.5° C. to about 35.5° C., and introducing rVSV into the suspension cell culture medium at a multiplicity of infection (MOI) rate of from about 0.5 to about 1.5 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 20 to 28 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of from about 36.8° C. to about 37.2° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is an ebola virus glycoprotein, optionally comprising the amino sequence of SEQ ID NO: 3 or SEQ ID NO: 4; (c) reducing the temperature of the suspension cell culture medium to from about 33° C. to about 35° C., and introducing rVSV into the suspension cell culture medium at a multiplicity of infection (MOI) rate of from about 0.8 to about 1.2 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 22 to 26 hours post infection, wherein the viral envelope glycoprotein in incorporated into the viral envelope of the isolated rVSV.

In one embodiment, the present disclosure provides a method of producing replication-incompetent recombinant vesicular stomatitis virus (rVSV), comprising: (a) inoculating a suspension cell culture medium with a plurality of HEK293F cells, wherein the suspension cell culture medium is at a temperature of 37° C.±0.1° C.; (b) transfecting the HEK293F cells in the suspension cell culture medium with a transfection reagent (e.g., a polyethylenimine transfection reagent) and a plasmid comprising a nucleic acid molecule encoding a viral envelope glycoprotein to produce a population of packaging cells expressing the viral envelope glycoprotein, wherein the viral envelope glycoprotein is an ebola virus glycoprotein, optionally comprising the amino sequence of SEQ ID NO: 3 or SEQ ID NO: 4; (c) reducing the temperature of the suspension cell culture medium to about 34° C.±0.1° C. and introducing rVSV into the suspension cell culture medium at a multiplicity of infection (MOI) rate of about 1 to infect the population of HEK293F cells expressing the viral envelope glycoprotein, wherein the rVSV has been engineered such that the rVSV does not express a functional envelope glycoprotein; and (d) isolating rVSV produced from the population of packaging cells from 23 to 25 hours post infection, wherein the viral envelope glycoprotein in incorporated into the viral envelope of the isolated rVSV.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Production of High Titer Replication Incompetent Recombinant Vesicular Stomatitis Virus (rVSV) Using Suspension HEK293 Cells in a Chemically Defined Cell Culture Medium

A replication incompetent form of rVSV that was absent of glycoprotein gene was produced at high titer using a suspension cell line using the production scheme illustrated in FIG. 1. Specific harvest timing was determined to reduce negative impact of residual transfection reagent on subsequent steps to eliminate the need for media exchange prior to infection, thus significantly improving scalability of the process.

The materials included:
Cell line: Gibco CTS Viral Production Cells (HEK293F derived suspension cell line)
Cell Culture Medium: Gibco CTS LV-MAX Production Medium
Plasmid: pMD2.G
Transfection Reagent: PEI MAX (40 kDa)
Stock Virus for Infection: VSV^{ΔG:Fluc/GFP}

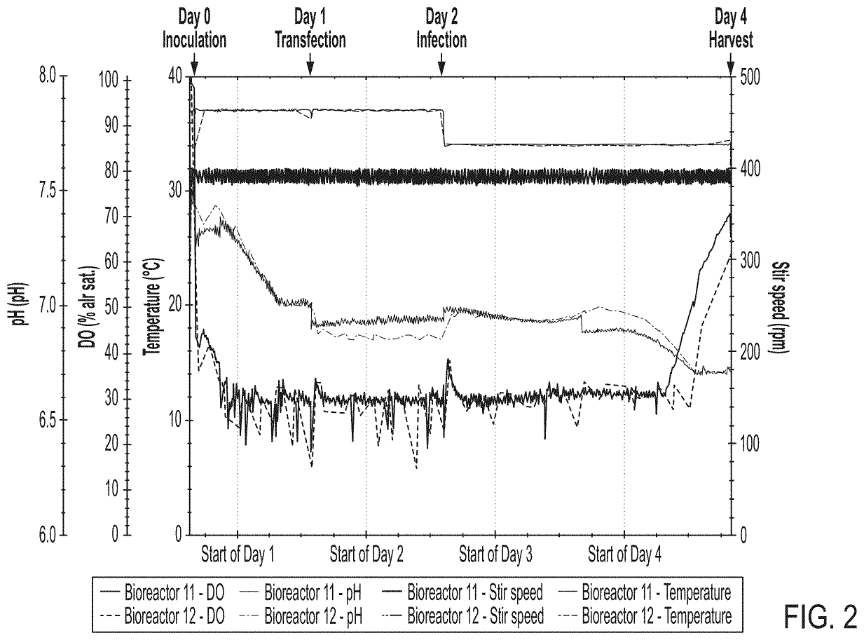

On day 0, viral production cells were seeded at 1.7 E6/mL in LV-MAX production medium in a 125 mL shake flask. The shake flask was incubated at 37° C. with 8% CO₂ and a rocker speed of 125 rpm. A comparable experiment was performed in a 250 mL bioreactor (AmbrO 250 system, Sartorius AG) at 200 mL working volume using a temperature setpoint of 37° C., pH of 7.0±0.3, dissolved oxygen level of 30%, agitation power input of 22 W/m³, air sparge rate of 0.01 volume of air per volume of liquid per minute (VVM) and air overlay of 0.02 VVM, as shown in Table 1, below, and in FIG. 2.

TABLE 1
Bioreactor Parameters
Transfection Parameters:

| Transfection Cell Density | 3.0 × 10E6 cells/mL |
|---|---|
| Transfection Reagent | PEI MAX |
| DNA Concentration | 1.5 μg/ml |
| PEI:DNA ratio | 2.5 |

Infection Parameters:

| | |
|---|---|
| Media Exchange | No |
| MOI | 0.01 |

Bioreactor Parameters:

| | |
|---|---|
| Temperature | 37.0° C. → 34° C. |
| pH | 7.0 ± 0.3 (DB) |
| DO | 30% |
| Agitation | 390 rpm (22 W/m3) |
| Air Overlay | 4 ccm |
| Air Sparge | 2 ccm |
| O₂ Sparge | DO control |
| CO₂ Sparge | pH control |
| Base Addition | pH control |
| Glucose Feeding | Feed up to 5 g/L when residual < 2.5 g/L |

On day 1, cells were grown to a target cell density of 3.0 E6/mL. The pMD2.G plasmid was diluted in LV-MAX production medium to reach 2.5% of post transfection cell culture volume and at a final DNA concentration of 1.5 μg/mL. PEI MAX was diluted in LV-MAX production medium to reach 2.5% of post transfection cell culture volume and a PEI MAX to plasmid ratio of 2.5:1. The transfection mix was prepared by adding diluted PEI MAX to diluted plasmid. The transfection mix was gently swirled, and then incubated for 10 minutes at room temperature prior to addition to the shake flask.

On day 2, the cells were counted to determine the viable cell density. The VSV stock virus was thawed on ice. The stock virus was diluted 1000-fold with cold LV-MAX production medium, and the diluted stock virus was spiked directly into the cell culture using a multiplicity of infection (MOI) of 0.01. Immediately following infection, the temperature of the incubator or bioreactor was lowered to 34° C.

For production in the bioreactor, from day 2 onward, glucose was fed daily up to 5 g/L when residual glucose dropped to <2.5 g/L.

On day 4, approximately 48 hours post infection (hpi), the crude cell culture was centrifuged at 3000×g at 4° C. for 15 minutes to clarify and remove cell debris, and the purified VSV material was stored at −80° C. until further use.

The process discussed above yielded an infectious titer of ˜5E8-1E9 pfu/mL, which is comparable to results achieved with adherent cell production systems. Both shake flask and bioreactor experiments yielded comparable titers.

### Example 2: Evaluation of Varying Transfection, Infection, Culture and Harvest Parameters on Viral Titer in the Production of rVSV in Suspension Cell Culture

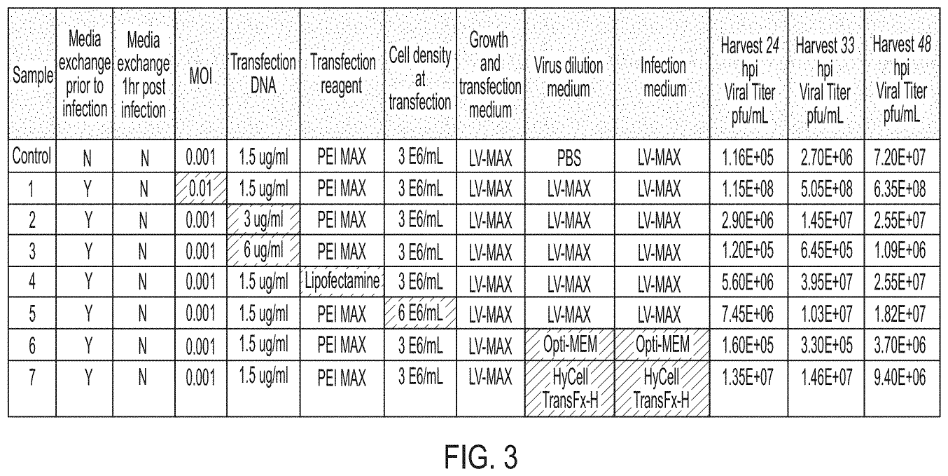

Shake flask experiments were performed as discussed in Example 1 with variations in parameters to evaluate the effect on viral titer. Multiplicity of infection (MOI) rate, quantity of transfection DNA, transfection reagent, cell density at transfection, and virus dilution and infection medium were evaluated in a first set of experiments, as shown in FIG. 3. Among the varying parameters tested, MOI rate was identified as having the most significant impact on viral titer at 24, 33 and 48 hours post infection (see three columns at right side of FIG. 3).

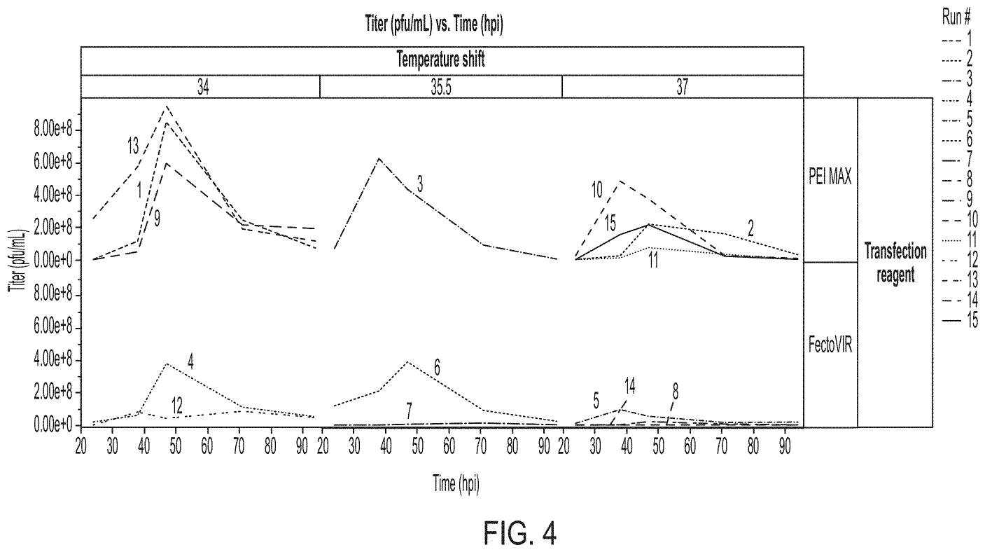

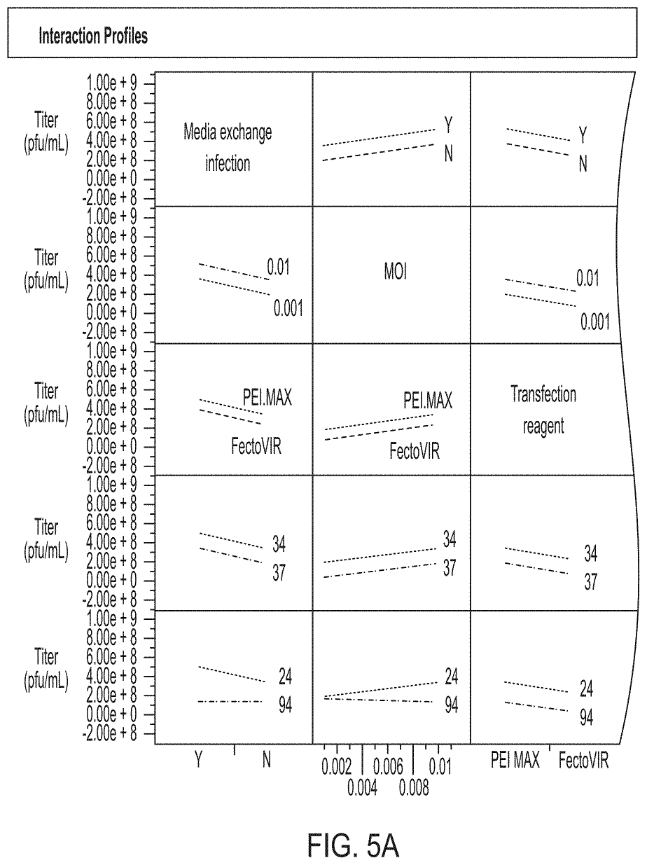

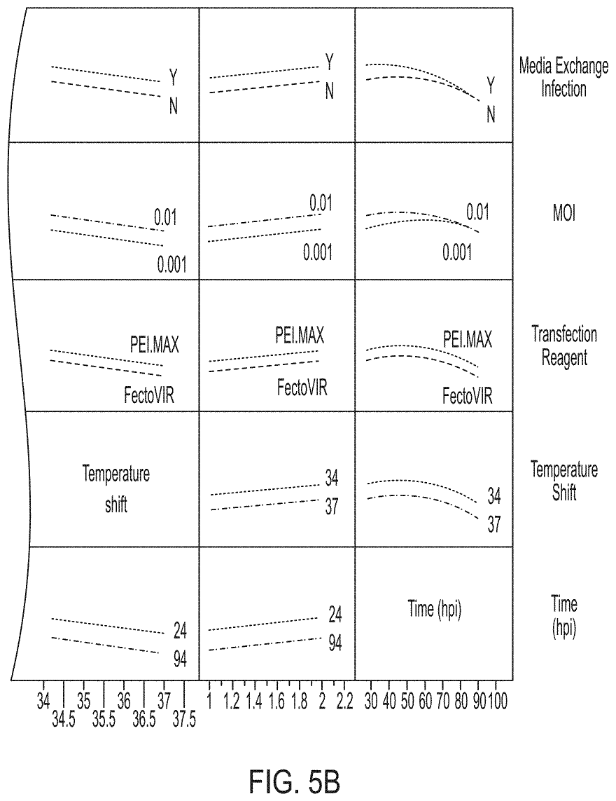

A second set of shake flask experiments was also performed as discussed in Example 1 with the varying parameters set forth in Table 2, below. The factors evaluated included the choice of transfection reagent (PEI MAX or FectoVIR-AAV), whether or not to perform media exchange prior to infection, a MOI rate ranging from 0.001 to 0.01, a temperature shift post infection ranging from 34° C. to 37° C. (from an original temperature of 37° C.), and harvest timing ranging from 24 hpi to 96 hpi (24, 36, 48, 72 and 96 hpi were evaluated). The resulting viral titer corresponding to each of these fifteen runs is illustrated in FIG. 4 as a function of transfection reagent (PEI MAX or FectoVIR), temperature shift, and harvest time (hpi). FIGS. 5A and 5B illustrates the interaction profiles of each of the various parameters relative to one another as a function of viral titer (pfu/mL) ranging from 2.78E8 to 4.89E8. As can be seen in FIGS. 5A and 5B, media exchange prior to infection and a higher MOI (0.01) was preferred at 24 hpi, but effects on viral titer became less significant at later harvest time points. Based on the interaction profiles illustrated in FIGS. 5A and 5B, media exchange prior to infection was deemed unnecessary, and a harvest time of 44.4 hpi was preferred when coupled with a MOI rate of 0.01, a PEI MAX transfection reagent, and a temperature shift from 37° C. to 34° C.

TABLE 2
Parameters Corresponding to 15 Runs for Production of rVSV-ΔG

| Run | Media Exchange | | Transfection | Temperature |
|---|---|---|---|---|
| # | Prior to Infection | MOI | Reagent | Shift |
| 1 | N | 0.01 | PEI MAX | 34 |
| 2 | Y | 0.001 | PEI MAX | 37 |
| 3 | Y | 0.0055 | PEI MAX | 35.5 |
| 4 | Y | 0.001 | FectoVIR | 34 |
| 5 | Y | 0.0055 | FectoVIR | 37 |
| 6 | Y | 0.01 | FectoVIR | 35.5 |
| 7 | N | 0.001 | FectoVIR | 35.5 |
| 8 | N | 0.001 | FectoVIR | 37 |
| 9 | N | 0.001 | PEI MAX | 34 |
| 10 | Y | 0.01 | PEI MAX | 37 |
| 11 | N | 0.0055 | PEI MAX | 37 |
| 12 | N | 0.0055 | FectoVIR | 34 |
| 13 | Y | 0.01 | PEI MAX | 34 |
| 14 | N | 0.01 | FectoVIR | 37 |
| 15 | N | 0.01 | PEI MAX | 37 |

### Example 3: Production of High Titer Replication Incompetent Pseudotyped Recombinant Vesicular Stomatitis Virus (rVSV) Using Suspension HEK293 Cells in a Chemically Defined Cell Culture Medium

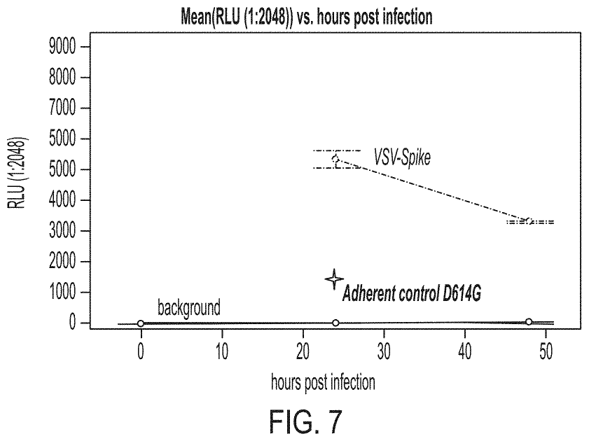

A pseudotyped replication incompetent form of rVSV that was absent of glycoprotein gene was produced at high titer using a suspension cell line using the production scheme illustrated in FIG. 7. The production followed that detailed in Example 1, except that the multiplicity of infection (MOI) was increased from 0.01 to 1, and the harvest time was decreased from approximately 48 hours post infection (hpi) to approximately 24 hpi.

The process yielded an infectious titer of more than 3-fold greater than that produced from a comparable adherent cell production system (making the same rVSV). The titer was measured using a luciferase assay in Vero cells, and the results are shown in FIG. 7. Briefly, for titering the pseudotyped virus, Vero cells were infected with the pseudotyped virus with certain dilution ratio and luciferase signal was read at 24 hours post infection where titer is reported as a number of RLU/mL (RLU, relative light unit).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. A method of producing replication-incompetent recombinant vesicular stomatitis virus (rVSV), comprising:
(a) inoculating a suspension cell culture medium with a plurality of packaging cells;
(b) transfecting the packaging cells in the suspension cell culture medium with a plasmid comprising a nucleic acid molecule encoding a viral envelope glycoprotein to produce a population of packaging cells expressing the viral envelope glycoprotein;
(c) introducing rVSV into the suspension cell culture medium at a multiplicity of infection (MOI) rate of from 0.001 to 3 to infect the population of packaging cells expressing the viral envelope glycoprotein, wherein the rVSV has been engineered such that the rVSV does not express a functional envelope glycoprotein; and
(d) isolating rVSV produced from the population of packaging cells from 15 to 65 hours post infection, wherein the viral envelope glycoprotein in incorporated into the viral envelope of the isolated rVSV.

2. The method of claim 1, wherein the MOI is from 0.001 to 1.0.

3. The method of claim 1, wherein the MOI is from 0.005 to 0.05.

4. The method of claim 1, wherein the MOI is from 0.008 to 0.012.

5. The method of claim 1, wherein the MOI is about 0.01.

6. The method of claim 1, wherein the MOI is from 0.5 to 1.5.

7. The method of claim 1, wherein the MOI is from 0.8 to 1.2.

8. The method of claim 1, wherein the MOI is about 1.

9. The method of any one of claims 1-5, wherein the rVSV produced from the packaging cells is isolated from 40 to 50 hours post infection.

10. The method of any one of claims 1-5, wherein the rVSV produced from the packaging cells is isolated from 43 to 47 hours post infection.

11. The method of any one of claim 1, 2 or 6-8, wherein the rVSV produced from the packaging cells is isolated from 20 to 30 hours post infection.

12. The method of any one of claim 1, 2 or 6-8, wherein the rVSV produced from the packaging cells is isolated from 23 to 25 hours post infection.

13. The method of any one of claims 1-12, wherein the packaging cells are mammalian cells.

14. The method of claim 13, wherein the packaging cells are primate cells or human cells.

15. The method of claim 14, wherein the packaging cells are human embryonic kidney (HEK) cells.

16. The method of claim 15, wherein the HEK cells are HEK293 cells.

17. The method of claim 16, wherein the HEK293 cells are HEK293F cells, HEK293T cells, HEK293SF cells, or HEK293S cells.

18. The method of claim 17, wherein the HEK293 cells are HEK293F cells.

19. The method of any one of claims 1-18, wherein the suspension cell culture medium is a serum-free and protein-free medium.

20. The method of any one of claims 1-19, wherein the isolated rVSV is produced at an infectious titer of at least 1×10⁶ plaque forming units (pfu)/mL.

21. The method of claim 20, wherein the isolated rVSV is produced at an infectious titer of at least 1×10⁷ pfu/mL.

22. The method of claim 21, wherein the isolated rVSV is produced at an infectious titer of at least 1×10⁸ pfu/mL.

23. The method of claim 22, wherein the isolated rVSV is produced at an infectious titer of at least 5×10⁸ pfu/mL.

24. The method of claim 23, wherein the isolated rVSV is produced at an infectious titer of at least 1×10⁹ pfu/mL.

25. The method of any one of claims 1-19, wherein the isolated rVSV is produced at an infectious titer at least comparable to a titer produced by an adherent cell culture control that produces the same rVSV.

26. The method of claim 25, wherein the isolated rVSV is produced at an infectious titer at least two fold greater than the titer produced by the adherent cell culture control that produces the same rVSV.

27. The method of claim 26, wherein the isolated rVSV is produced at an infectious titer at least three fold greater than the titer produced by the adherent cell culture control that produces the same rVSV.

28. The method of any one of claims 1-27, wherein the viral envelope glycoprotein is a vesicular stomatitis virus G protein.

29. The method of any one of claims 1-27, wherein the viral envelope glycoprotein is a class I fusion protein.

30. The method of any one of claims 1-27, wherein the viral envelope glycoprotein is a class II fusion protein.

31. The method of any one of claims 1-27, wherein the viral envelope glycoprotein is a class III fusion protein.

32. The method of any one of claims 1-27, wherein the viral envelope glycoprotein is a coronavirus spike protein.

33. The method of claim 32, wherein the coronavirus spike protein is a spike protein of SARS-CoV-2.

34. The method of claim 33, wherein the coronavirus spike protein comprises the amino acid sequence of SEQ ID NO: 1.

35. The method of claim 33, wherein the coronavirus spike protein comprises the amino acid sequence of SEQ ID NO: 2.

36. The method of any one of claims 1-27, wherein the viral envelope glycoprotein is an ebola virus glycoprotein.

37. The method of claim 36, wherein the ebola virus glycoprotein comprises the amino acid sequence of SEQ ID NO: 3 or SEQ ID NO: 4.

38. The method of any one of claims 1-27, wherein the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses.

39. The method of any one of claims 1-38, wherein transfecting the packaging cells comprises use of a polyethylenimine transfection reagent.

40. The method of any one of claims 1-38, wherein transfecting the packaging cells comprises uses of a lipofectamine transfection reagent or FectoVIR-AAV-transfection reagent.

41. The method of any one of claims 1-40, wherein the cell culture medium comprises a cell density of at least 3×10⁶ packaging cells/mL when transfecting the packaging cells.

42. The method of any one of claims 1-41, wherein transfecting the packaging cells comprises use of a DNA concentration of at least 1.5 μg/mL.

43. The method of any one of claims 1-42, wherein transfecting the packaging cells comprises use of a transfection reagent:DNA ratio of from about 2 to about 3.

44. The method of claim 43, wherein transfecting the packaging cells comprises use of a transfection reagent:DNA ratio of about 2.5.

45. The method of any one of claims 1-44, wherein the suspension cell culture medium is maintained at a temperature during inoculation and transfection, and the temperature of the suspension cell culture medium is reduced to a lower temperature when introducing rVSV into the suspension cell culture medium.

46. The method of claim 45, wherein the temperature is about 37° C.

47. The method of claim 46, wherein the reduced temperature is from about 32° C. to about 36.5° C.

48. The method of claim 47, wherein the reduced temperature is about 35.5° C.

49. The method of claim 47, wherein the reduced temperature is about 34° C.

50. The method of any one of claims 1-49, wherein the suspension cell culture medium is not exchanged prior to introducing rVSV into the suspension cell culture medium.

51. A method of producing replication-incompetent recombinant vesicular stomatitis virus (rVSV), comprising:
(a) inoculating a suspension cell culture medium with a plurality of HEK293 cells, wherein the suspension cell culture medium is at a temperature of about 37° C.;
(b) transfecting the HEK293 cells in the suspension cell culture medium with a polyethylenimine transfection reagent and a plasmid comprising a nucleic acid molecule encoding a viral envelope glycoprotein to produce a population of packaging cells expressing the viral envelope glycoprotein;
(c) reducing the temperature of the suspension cell culture medium to about 34° C. and introducing rVSV into the suspension cell culture medium at a multiplicity of infection (MOI) rate of about 0.01 to infect the population of HEK293 cells expressing the viral envelope glycoprotein, wherein the rVSV has been engineered such that the rVSV does not express a functional envelope glycoprotein; and
(d) isolating rVSV produced from the population of packaging cells from 40 to 48 hours post infection, wherein the viral envelope glycoprotein in incorporated into the viral envelope of the isolated rVSV.

52. A method of producing replication-incompetent recombinant vesicular stomatitis virus (rVSV), comprising:
(a) inoculating a suspension cell culture medium with a plurality of HEK293 cells, wherein the suspension cell culture medium is at a temperature of about 37° C.;
(b) transfecting the HEK293 cells in the suspension cell culture medium with a polyethylenimine transfection reagent and a plasmid comprising a nucleic acid molecule encoding a viral envelope glycoprotein to produce a population of packaging cells expressing the viral envelope glycoprotein;
(c) reducing the temperature of the suspension cell culture medium to about 34° C. and introducing rVSV into the suspension cell culture medium at a multiplicity of infection (MOI) rate of about 1 to infect the population of HEK293 cells expressing the viral envelope glycoprotein, wherein the rVSV has been engineered such that the rVSV does not express a functional envelope glycoprotein; and
(d) isolating rVSV produced from the population of packaging cells from 20 to 28 hours post infection, wherein the viral envelope glycoprotein in incorporated into the viral envelope of the isolated rVSV.

53. The method of claim 51 or 52, wherein the viral envelope glycoprotein is a class I fusion protein, a class II fusion protein, or a class III fusion protein.

54. The method of claim 51 or 52, wherein the viral envelope glycoprotein is a coronavirus spike protein.

55. The method of claim 54, wherein the coronavirus is SARS-CoV-2.

56. The method of claim 51 or 52, wherein the viral envelope glycoprotein is an ebola virus glycoprotein.

57. The method of claim 51 or 52, wherein the viral envelope glycoprotein is a viral envelope glycoprotein of a virus selected from the group consisting of flaviviruses, alphaviruses, togaviruses, coronavirues, herpesviruses, hepadnaviruses, poxviruses, paramyxoviruses, rhabdoviruses, bunyaviruses, filoviruses, retroviruses, hepatitis viruses, influenza A viruses, and influenza B viruses.

58. The method of any one of claims 51-57, wherein the cell culture medium comprises a cell density of at least 3×10⁶ HEK293 cells/mL when transfecting the HEK293 cells.

59. The method of any one of claims 51-58, wherein transfecting the HEK293 cells comprises use of a DNA concentration of at least 1.5 μg/mL.

60. The method of any one of claims 51-59, wherein transfecting the HEK293 cells comprises use of a transfection reagent:DNA ratio of about 2.5.

61. The method of any one of claims 51-60, wherein the HEK293 cells are HEK293F cells, HEK293T cells, HEK293SF cells, or HEK293S cells.

62. The method of claim 61, wherein the HEK293 cells are HEK293F cells.
